(19) United States
(12) Patent Application Publication
(10) Pub. No.: US 2006/0025494 A1
(43) Pub. Date: Feb. 2, 2006

(54) Denture adhesive

(76) Inventor: Robert C Gasman, West Windsor, NJ (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/539,336

(22) PCT Filed: Dec. 17, 2003
(86) PCT No.: PCT/US03/40658

§ 371 Date: Jun. 15, 2005

Related U.S. Application Data
(60) Provisional application No. 60/435,058, filed on Dec. 20, 2002.

(51) Int. Cl. A61K 6/00

(52) U.S. Cl. 523/120

(57) ABSTRACT

Denture adhesive compositions with sufficient initial adhesion to the gums that build up enough cohesive strength upon hydration with saliva to withstand the stresses occurring during food mastication and cushions the gums from contact with the denture during chewing. The adhesive comprises a mixed partial salt of an amino ethyl ethylene urea substituted polyalkylvinylether/maleic acid copolymer wherein the cations of the salt are selected from the group consisting of zinc, calcium, magnesium, strontium, zirconium, sodium, and potassium.

### FIELD OF THE INVENTION

This invention relates to denture adhesives and to methods for making and using denture adhesives. In particular, this invention relates to an amino ethyl ethylene urea-substituted mixed partial salt of a copolymer of a polyalkyl vinyl ether and maleic acid, (PVE/MA) as a new denture adhesive active.

### BACKGROUND OF THE INVENTION

Ordinary removable dentures and dental plates function as a substitute for all or part of missing teeth ordinarily found in the mouth. While dentures are usually carefully fitted for the user, the fit can change over time, causing discomfort and slippage. In order to alleviate the discomfort and to control the slippage, a denture adhesive may be applied to the denture. The denture adhesive acts both as an adherent and as a gasket or cushion between the dentures and the gums of the denture wearer. Denture adhesives are known and can be in the form of creams, liquids, powders, and liners, either extruded or with a non-adhesive self-supporting layer.

Denture adhesives are designed to provide a diverse range of seemingly contradictory properties in an extraordinarily complex environment, i.e., the human mouth. Denture adhesives must develop a quick tack upon contact with the oral mucosa to prevent the denture from coming loose; they must also develop cohesive strength quickly when hydrated with moisture or saliva. They must also hold the denture in place for a period of time, which may be as long as 12 to 16 hours. Denture adhesives should only need to be applied once per day and should be nontoxic and organoleptically acceptable. They must not be easily washed out, i.e., the adhesive must resist degradation under extreme environmental changes occurring in the oral cavity during the course of daily activities, e.g., drinking hot or cold beverages. But, denture adhesives must also easily release from the dentures and oral mucosa after use.

Early denture adhesives contained finely ground particles of natural gums that expanded when wet with water to become a viscous gel, which acted as a cushion and an adherent between the dental plate and the gum tissue. These early denture adhesives have been supplanted by polymeric denture adhesives.

Several attempts have been made to fashion a denture adhesive using various polymer salts. One preferred polymer from the field is a mixed partial salt of a copolymer of maleic acid and alkyl vinyl ether, sold under the GANTREZ® trade name. This class of polymers was described as a possible denture adhesive in Germann, et al. U.S. Pat. No. 3,003,988, which issued more than 30 years ago.

That patent describes synthetic, water sensitized but water insoluble, materials comprising mixed partial salts of lower alkyl vinyl ether-maleic acid copolymers for stabilizing dentures. The salts mentioned in the patent are a mixture of (a) calcium and (b) sodium, or potassium or quaternary ammonium compounds in a 1:1 to 5:1 molar ratio. The calcium hydroxide and monovalent cation hydroxides are added to an aqueous dispersion of the acid copolymer to form a mixed salt.

The use of this class of materials has been described in a variety of other patents. Examples include U.S. Pat. Nos. 5,830,933; 4,980,391; 4,373,036; 5,006,571; 4,521,551; 3,868,432 and European Published Patent Application No. 406,643.

In order to provide additional adhesive and cohesive properties to denture adhesives made from GANTREZ® polymers, one approach has been to manipulate the salt form of the copolymer. Examples can be found in WO 92/22280, WO 92/10988, WO 92/10987, and WO 92/10986.

One approach is found in U.S. Pat. No. 4,758,630 to Shah et al., issued Jul. 19, 1988, which is directed to denture adhesives having mixed partial salts of zinc and strontium.

Another approach is reported in U.S. Pat. No. 5,073,604 to Holeva et al., issued Dec. 17, 1991, which is directed to a denture adhesive made from a partial salt of a GANTREZ® copolymer, wherein the cations are zinc ions in combination with calcium or strontium ions in combination with with calcium, and optionally containing sodium cations.

U.S. Pat. No. 5,298,534 to Prosise et al., issued Mar. 29, 1994, reports using GANTREZ® salts of calcium, sodium, strontium, zinc, magnesium and potassium with boron cross-linked guar gum and an oil base as a carrier. The preferred mixed salt is a Ca/Na mixed salt. The guar gum is “critical” to the asserted extended holding power and viscosity building properties.

Another approach has been to employ an adhesion adjuvant in the formulation or converting the copolymer into a terpolymer, and examples of these approaches can be found in U.S. Pat. Nos. 3,736,274, 5,037,924 and 5,093,387.

Despite the efforts which have been put into improving the properties of maleic anhydride/alkyl vinyl ether type polymers and their salts as useful denture adhesive actives, these formulations do not provide the full desired measure of adhesion, cohesion, agreeable taste and resistance to washout from beneath the denture. Therefore, there exists a need for improved denture adhesive formulations.

### SUMMARY OF THE INVENTION

The principal object of the present invention therefore, is to provide a new and improved denture adhesive composition having an unusually strong affinity for oral tissues and for the acrylic denture, and builds up a cohesive strength for good hold characteristics when hydrated with moisture or saliva so as to be able to resist stresses such as those that occur upon mastication.

Another object of the invention is to provide an adhesive in which the improved adhesive retains its adhesive properties for prolonged periods of time and resists wash out. Still another object of the invention is to provide an adhesive that cushions the gums from the denture stresses that develop during the chewing of food.

Additional objects and advantages of the invention will be set forth in part in the description that follows, and in part will be obvious from this description, or may be learned by practice of the invention. The objects and advantages of the invention may be realized and attained by means of the instrumentalities and combinations particularly pointed out in the appended claims.

To achieve the foregoing objects and in accordance with the purpose of the invention, as embodied and broadly described herein, the invention provides a denture adhesive comprising a mixed metal salt containing sodium, and/or calcium, and/or zinc and/or strontium and/or magnesium, and/or potassium cations, of an AEEU-substituted-partial salt of polyalkyl vinyl ether/maleic acid, copolymer.

To further achieve the foregoing objects and in accordance with the purpose of the invention, the invention further provides a method for securing a removable dental device in the mouth comprising applying a denture adhesive comprising a mixed metal, AEEU-substituted-partial salt of polyalkylvinyl ether, maleic acid, copolymer. The function of the AEEU substituent in this salt copolymer is to confer on this salt copolymer the property of specific adhesion to the oral tissues and to an acrylic denture.

### DETAILED DESCRIPTION OF THE INVENTION

### BRIEF DESCRIPTION OF THE DRAWINGS

While this specification concludes with claims particularly pointing out and distinctly claiming that which is regarded as the present invention, the objects and advantages of this invention may be more readily ascertained from the following description of a preferred embodiment when read in conjunction with the accompanying drawings.

FIG. 1 is a chart depicting a dislodge force profile comparison of three denture adhesives creams containing varying adhesive components: (1) Gantrez Salt 418, (2) AEEU substituted at a level of 0.3 molar substitution to Salt 418 and (3) AEEU substituted Salt 418 with the addition of 0.5% S-97 Gantrez acid.

The polymer salts of the present invention are the mixed partial salts of a C₁-C₄ alkyl vinyl ether maleic acid copolymer. Methyl vinyl ether/maleic anhydride (MVE/MA) which has a specific viscosity larger than 1.2 in methyl ethyl ketone at 25° C., is the preferred starting material copolymer. The anhydride copolymer is mixed with the AEEU at room temperature and then converted to an amide at elevated temperatures, hydrolized to the acid and then neutralized with the appropriate cations. Most preferably, copolymers are selected from the copolymers set forth in U.S. Pat. No. 5,525,652, the disclosure of which is incorporated herein by reference. While the copolymers of that invention are the preferred copolymers, this invention is not limited to the use of those copolymers.

Suitable metal salt combinations include, but are not limited to, a mixed salt of sodium/magnesium/zinc, a mixed salt of magnesium/zinc, a mixed salt of zinc/calcium, a mixed salt of calcium/sodium, a mixed salt of calcium/potassium, a mixed salt of calcium/strontium, a mixed salt of calcium/zirconium, a mixed salt of magnesium/strontium, a mixed salt of magnesium/zirconium, a mixed salt of sodium/strontium, a mixed salt of sodium/zirconium, a mixed salt of zinc/strontium, a mixed salt of zinc/zirconium and a mixed salt of magnesium/sodium and the like.

The degree of substitution limits for alkyl vinyl ether/maleic acid (MVE/MA) partial salts of the invention are: (a) from about 5% to about 55% magnesium, more preferably from about 15% to about 45% magnesium, and most preferably from about 20% to about 40% magnesium; (b) from about 5% to about 65% zinc, more preferably from about 10% to about 60% zinc, and most preferably from about 15% to about 55% zinc; and (c) from about 0% to about 40% sodium, preferably from about 0% to about 35% sodium, and most preferably from about 0% to about 30% sodium.

The polymer salt is prepared by the partial neutralization of an aqueous premix of AEEU and the alkylvinylether/maleic acid or anhydride copolymer with the oxides and hydroxides of the alkali salts, for example, calcium, strontium, potassium, magnesium, zinc, sodium, and zirconium. When the salt is prepared, the metal compounds used, react with the carboxylic acid groups on the copolymer and neutralize them. Preferably less than 100% of the carboxylic acid groups on the copolymer chain are neutralized. More preferably, the metal compounds neutralize from the 20% to about 95% of the carboxylic acid groups of the copolymer and most preferably from about 30% to about 85% of the carboxylic acid groups.

In choosing the alkaline, inorganic metal compound used to neutralize the carboxylic acid groups, the anion portion of the compound is restricted, and is preferably an oxide, hydroxide, or carbonate. In general, the oxides or hydroxides are preferred because of their ease of handling, availability and the generally innocuous nature of the by-products formed in their reaction with carboxylic acids.

To make the salt of the invention, an aqueous dispersion of the methylvinylether/maleic anhydride Copolymer and an aqueous solution of AEEU are first blended together at 25° C. and held at 25° C. for 20 minutes with continuous mixing, after that 20 minutes an aqueous dispersion of the metal oxides and or hydroxides is added to the AEEU, anhydride, water mixture and then the temperature is gradually raised. The dispersion of metal compounds and the AEEU-anhydride copolymer dispersion are combined and allowed to react at an elevated temperature, preferably between 50° C. and 90° C. The product salt formed is dried, either in a tray or drum drier and preferably milled to less than about 100 mesh, then dispersed in a pharmacologically acceptable carrier to form the denture adhesive of this invention using techniques well known in the art.

MVE/MA copolymer is available from ISP Corporation in New Jersey, under the trade name GANTREZ®. The polymer is available in a GANTREZ-S series which is methyl vinyl ether/maleic acid and in GANTREZ AN series, which is methyl vinyl ether/maleic anhydride.

AEEU, which has the general structure: 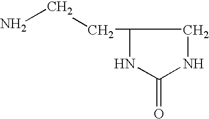
is available from King Industries, Norwalk, Conn. under the tradename K-Flex XM-3323.

The denture adhesive composition of the present invention contains a denture adhesive effective amount of the AEEU-substituted denture adhesive copolymer salt in a pharmacologically acceptable carrier. The AEEU-substituted denture adhesive copolymer salt usually comprises from about 15% to about 55% by weight of the denture adhesive composition. Preferably it is about 25% to about 35% by weight of the denture adhesive cream composition, and most preferably from about 27% to about 33% by weight of the composition in a cream formulation and about 45% to about 55% by weight in a powder formulation.

Denture adhesives of this invention can be formulated by conventional techniques, in the form of a powder, a liquid, a cream, or a liner. The compositions also contain other active and non-active ingredients.

When the denture adhesive is formulated into a liner, the liner may be a hot-melt extruded film as described in for example, U.S. Pat. No. RE33,093 to Schiraldi et al. and U.S. Pat. No. 6,375,963 to Repka et al., both incorporated by reference herein. In another embodiment, the denture adhesive may be in the form of a liner, wherein the denture adhesive composition is applied to a non-adhesive-self-supporting layer, as described in, for example, U.S. Pat. Nos. 4,632,880 and 4,503,116, to Lapidus and U.S. Pat. No. 5,880,172 to Rajaiah et al., and U.S. Pat. No. 5,872,160 to Liang, all of which are incorporated herein by reference.

The active ingredient in the adhesive is the inventive metal partial salt of the AEEU-substituted MVE/MA copolymer. The AEEU degree of substitution should be between 0.05% and 0.5%, but preferably between 0.1% and 0.4%. Without being bound by theory, it is believed that the strong hydrogen bonding between the ureido group in the AEEU molecule and the peptide linkages in the oral tissues and the carbonyl groups in the acrylic polymer of the denture is responsible for the strong specific adhesion of this AEEU substituted copolymer to the gums and acrylic denture.

In addition, the adhesive agent sodium carboxymethyl cellulose gum may be added as an active ingredient. Non-active ingredients may include petrolatum, mineral oil, flavors, colors, preservatives, thickeners and a non-toxic anti-caking agent such as fumed silica.

In addition to the denture adhesive, the composition contains a pharmacologically acceptable carrier. The pharmacologically acceptable carrier contains conventional materials and if desired, may contain any additional adhesive adjuvants that have been used conventionally in the art.

For instance, the carrier may contain a carboxymethyl cellulose gum that is used for sensitizing the adhesive to moisture. When present, the cellulose gum preferably comprises from about 10% to about 38% by weight of the denture adhesive cream composition, more preferably from about 15% to about 33% and most preferably from about 20% to about 28% of the composition for a cream formula. In a powder formula, the cellulose gum preferably comprises from about 45% to about 55% of the composition. If included in the inventive composition, the cellulose gum should be present as the full or partial alkali metal salt, preferably a sodium salt.

Non-active ingredients that may be present in the carrier portion of the denture adhesive composition include thickening agents such as petrolatum and waxes; mineral oil to provide lubricity; flavors such as synthetic flavor oils and/or oils derived from plants and fruits; colors suitable for food, drug and cosmetic use and known as FD&C colors; preservatives such as the parabens, benzoic acid, benzoates and the like; viscosity modifiers; and non-toxic and anti-caking agents such as silica, magnesium stearate and talc.

In a cream formulation, mineral oil preferably comprises from about 12% to about 17% by weight of the composition. Petrolatum is used as the thickening agent in the cream formulation by retarding the separation of the formula vehicle from the rest of the cream formula during product storage, and it preferably comprises from about 20% to about 50%, preferably from about 25% to about 45% by weight of the adhesive cream composition.

An illustrative composition of a denture adhesive cream formulation using an AEEU-substituted GANTREZ salt of the invention follows:

| | Cream Weight | Cream Weight |
|---|---|---|
| Ingredient | % | % |
| AEEU-substituted | 30% | 30% |
| (Ca/Na 0.37%) GANTREZ ® | | |
| Salt | | |
| CMC | 24% | 24% |
| Flavorant | | 0.32% |
| Petrolatum | 26.5% | 27.18% |
| Mineral Oil | 18% | 18% |
| Preservative | | |
| Colorants | | |
| Gantrez Acid | 1% | |
| Fumed Silica | 0.5% | 0.5% |

An illustrative composition of a denture adhesive specially formulated to be compatible with a tube container and a powder formulation, using a Ca/Na 0.37% AEEU-MVE/MA salt of this invention follows:

| | Powder |
|---|---|
| Ingredient | Weight % |
| AEEU-substituted | 49.84% |
| (Ca/Na 0.37%) | |
| GANTREZ ® salt | |
| CMC | 49.84% |
| Flavorant | 0.32% |
| Petrolatum | |
| Mineral Oil | |
| Preservative | |
| Colorants | |
| Fumed Silica | |
| AC Polyethylene 6A | |

The following examples are to be construed as merely illustrative and not a limitation on the scope of the invention in any way.

### EXAMPLE 1

A partial sodium, calcium AEEU salt of poly(methyl vinyl ether-co-maleic acid) (MVE/MA) was prepared by adding 3.6 g. of AEEU to 9750 g. of water at room temperature with stirring followed by adding 726.2 g. of poly(methyl vinyl ether-co-maleic anhydride) to the aqueous AEEU solution after the solution had mixed for 10 minutes. The poly(methyl vinyl ether-comaleic anhydride) was mixed in the AEEU solution at room temperature for 20 minutes. Next 251.6 g. of calcium hydroxide was charged into the polymeric anhydride/AEEU solution at room temperature. Next a pre-mix of 18.6 g. of sodium hydroxide dissolved in 2750 g. of room temperature water was added to the calcium hydroxide/polymethyl vinyl ether maleic anhydride/AEEU mix with stirring. Next the temperature of this mixture was raised gradually to 65° C. with stirring and then the temperature was held at 65° C. for an additional 30 minutes after which the product was discharged, dried in a forced air oven overnight at 65° C. and then the product was milled. Product assay showed that this partial salt contained 0.37% AEEU, 11.6% calcium, and 0.91% sodium and 9.2% water.

The AEEU substituted partial sodium, calcium salt of poly(methyl vinyl ether maleic acid) prepared as described above was used to make a laboratory batch of denture adhesive paste (Formula B below). A control batch of adhesive was also made using unsubstituted calcium, sodium MVE/MA salt as the active (Formula A below). A third batch of adhesive paste was made with 0.5% GANTREZ Acid activator and AEEU substituted PVM/MA salt according to Formula C presented below:

| | | | FORMULA C |
|---|---|---|---|
| | | | AEEU SALT |
| | FORMULA | FORMULA | WITH 0.5% |
| | A | B | GANTREZ ® ACID |
| | CONTROL | CONTROL | FORMULA |
| Calcium, Sodium | 30.0 | | |
| MVE/MA Salt | | | |
| AEEU Substituted | | 30.0 | 30.0 |
| Calcium, Sodium | | | |
| MVE/MA Salt | | | |
| Sodium | 24.0 | 24.0 | 24.0 |
| Carboxymethyl- | | | |
| cellulose | | | |
| Petrolatum | 27.5 | 27.5 | 27.5 |
| Mineral Oil | 18.0 | 18.0 | 18.0 |
| Fumed Silica | 0.5 | 0.5 | 0.5 |
| GANTREZ ® Acid | | | 0.5 |

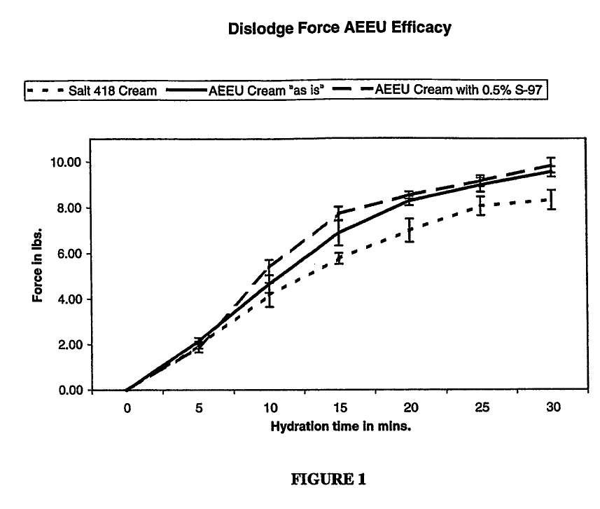

Paste adhesive formulas A, B, and C were tested for hang time (a measure of creep resistance), dislodge force, and subjective test performance according to the attached SOP's for these test procedures. The results of these tests are shown in FIG. 1.

The results indicate that formula C tested significantly higher for dislodge force than the control formula Both Formulas B and C tested significantly higher for hang time than Formula A. These results unexpectedly indicate that only a relatively small amount of AEEU is needed to produce a significant change in adhesive performance.

All publications, including, but not limited to, patents and patent applications cited in this specification, are herein incorporated by reference as if each individual publication were specifically and individually indicated to be incorporated by reference herein as though fully set forth.

The above description fully discloses the invention including preferred embodiments thereof. Modifications and improvements of the embodiments specifically disclosed herein are within the scope of the following claims. Without further elaboration it is believed that one skilled in the art can, given the preceding description, utilize the present invention to its fullest extent. Therefore any examples are to be construed as merely illustrative and not a limitation on the scope of the present invention in any way. The embodiments of the invention in which an exclusive property or privilege is claimed are defined as follows.

1. A denture adhesive composition comprising a denture adhesive effective amount of a denture adhesive salt which is an amino ethyl ethylene urea-substituted mixed partial salt of a copolymer of a polyalkyl vinyl ether and maleic acid, in a pharmaceutically acceptable carrier, wherein the cations of the salt are selected from, zinc, sodium, calcium, potassium, magnesium, strontium, zirconium or combinations thereof.

2. The composition according to claim 1, wherein the cations of the partial salt are a combination of sodium, magnesium and zinc.

3. The composition according to claim 1, wherein the cations of the partial salt are a combination of magnesium and zinc.

4. The composition according to claim 1, wherein the cations of the partial salt are a combination of zinc and calcium.

5. The composition according to claim 1, wherein the cations of the partial salt are a combination of calcium and sodium.

6. The composition according to claim 1, wherein the alkyl moiety is methyl.

7. The composition according to claim 1, wherein the composition is in the form of a cream.

8. The composition according to claim 6, wherein the AEEU-substituted denture adhesive copolymer salt comprises about 15-55% by weight of the total composition.

9. The composition according to claim 1, wherein the composition is in the form of a powder.

10. The composition according to claim 1, wherein the composition is in the form of a liner.

11. The composition according to claim 10, wherein the liner is in the form of a hot-melt extruded liner.

12. The composition according to claim 10, wherein the liner is in the form of a non-adhesive self-supporting layer.

13. The composition according to claim 1, further comprising a polyalkylvinylether/maleic acid activator.

14. The composition according to claim 13, where in the polyalkylvinylether/maleic acid activator is present from about 0.1% to about 10% by weight of the composition.

15. A compound which is a mixed partial salt of an amino ethyl ethylene urea substituted polyalkylvinylether/maleic acid copolymer.

16. The compound according to claim 15, wherein the acid copolymer is neutralized with a cation selected from the group consisting of zinc, sodium, calcium, potassium, magnesium, zirconium, and strontium, or combinations thereof.
